(19) United States
(12) Patent Application Publication
(10) Pub. No.: US 2012/0034178 A1
(43) Pub. Date: Feb. 9, 2012

(54) PIGMENT MIXTURES

(76) Inventors: Patrice Bujard, Courtepin (CH); Philippe Bugnon, Le Mouret (CH); Marc Mamak, New City, NY (US); Urs Leo Stadler, Madison, NJ (US)

(21) Appl. No.: 13/278,213

(22) Filed: Oct. 21, 2011

Related U.S. Application Data
(62) Division of application No. 12/520,592, filed on Jun. 22, 2009.

(51) Int. Cl. A61K 8/18; C09D 11/02; C08K 3/10; C04B 35/58; C08K 3/34; A61Q 1/02; C04B 35/46; C09D 1/00; C08K 3/22

(52) U.S. Cl. 424/63; 106/436; 106/439; 106/441; 106/442; 106/446; 106/417; 106/418; 524/406; 524/407; 524/409; 524/413; 524/449; 501/134; 501/1

(57) ABSTRACT

The present invention relates to novel pigment mixtures comprising at least two different particulate components A and B. The first component A comprises titanium suboxide(s), a titanium oxynitride or a titanium nitride species. Component B particle comprises inorganic, or organic pigments. The titanium suboxide particles display a bluish metallic luster, are new and form a further aspect of the present invention. When combined with particles from component B such as variously coated micas, for example, the bluish or bluish/black metallic color effect from component A leads to special color effects in the various mixtures.

This is a divisional of the U.S. application Ser. No. 12/520,592, filed Jun. 22, 2009, which is a national stage application which takes the benefit of international application No. PCT/EP2007/064017, filed Dec. 17, 2007.

The present invention relates to novel pigment mixtures comprising at least two different particulate components A and B. The first component A comprises titanium suboxide(s), a titanium oxynitride or a titanium nitride species. Component B particle comprises inorganic, or organic pigments. The titanium suboxide particles display a bluish metallic luster, are new and form a further aspect of the present invention. When combined with particles from component B such as variously coated micas, for example, the bluish or bluish/black metallic color effect from component A leads to special color effects in the various mixtures.

According to JP1072921 a superfine powder of anatase, rutile or brookite type TiO₂, preferably anatase type TiO₂ having 0.01-0.04 μm particle size is reduced by heating at 700-1,100° C. for about 1-2 hr in an atmosphere of gaseous hydrazine anhydride to obtain titanium oxide powder having a black color and particle size of 0.01-0.05 μm.

EP0478152B1 relates to a process for the preparation of titanium oxide having the formula TiOx in which x is less than 2, the process comprising heating a titanium oxide in the presence of carbon and in an atmosphere of a reducing gas and in the presence of carbon, characterized in that the titanium oxide starting material is placed on the surface of a support which is in the form of a sheet or board and which contains carbon, to produce substantially uncontaminated reduced oxide.

According to JP6115938 titanium dioxide is reduced with a mixed gas of hydrogen and titanium tetrachloride to give on low-order oxide of titanium. The reduction is preferably carried out by using a reducing gas having a ratio of hydrogen/titanium tetrachloride=2-500 (molecular ratio) at 600-1,400° C.

U.S. Pat. No. 5,320,782 relates to an electrically conductive titanium oxide composition having a low surface resistivity comprising: a capsule of silicon dioxide having a thickness of 5 to 100 Å; and at least one compound selected from the group consisting of titanium suboxides and titanium oxynitride encapsulated within said capsule, said titanium suboxides having the general formula TiOₓ, x being a positive real number less than 2, said titanium oxynitride having the general formula TiON, and said at least one compound being retained by said capsule in an acicular or platy form having an average largest dimension in the range of 0.1 to 20 μm and an aspect ratio of not less than 3; said titanium oxide composition being produced by the process comprising the steps of: forming a slurry comprising acicular or platy titanium oxide; adjusting a pH of the slurry to 9.5 to 11.0; adding a silicon compound to form a capsule of silicon dioxide which encapsulates said acicular or platy titanium oxide; and reducing said acicular or platy titanium oxide in said capsule to produce said at least one compound encapsulated in said capsule.

U.S. Pat. No. 5,985,020 relates to a platelet-shaped titanium dioxide reduction pigment comprising titanium dioxide, a titanium suboxide and optionally a further metal oxide or titanium oxynitride, obtained by solidifying an aqueous solution of a thermally hydrolyzable titanium compound on a continuous belt, detaching the resulting layer, coating the resulting titanium dioxide platelets, without drying in between, with further titanium dioxide by a wet method, drying optionally calcining the material, and optionally treating the material obtained with a reducing agent in a nonoxidizing gas atmosphere; and to a platelet-shaped titanium dioxide reduction pigment comprising titanium dioxide, at least one titanium suboxide and optionally a further metal oxide or titanium oxynitride, having a planeparallel surface, a thickness tolerance of lower than 10% and a layer thickness of lower than 500 nm. The platelet-shaped titanium dioxide reduction pigment is used in paints, printing inks, plastics, cosmetics or glazes for ceramics and glass, optionally as mixtures with commercially available pigments. The titanium dioxide platelets have a thickness of between 10 nm and 500 nm, preferably between 40 and 150 nm. The extent in the two other dimensions is between 2 and 200 μm and, in particular, between 5 and 50 μm.

US2004136898 relates to a coating powder based on chemically modified titanium suboxides having a defined defect structure, wherein the powder is modified by at least one metallic alloying element and described by the general formula Tiₙ₋₂Me₂O₂ₙ₋₁.

Reduced titania-coated luster pigments whose TiO₂ coating comprises or has been wholly converted to reduced titanium species (oxidation state of the titanium: from <4 to 2) have long been known as “dark pearl luster pigments” for the blue to black hue range.

For example, U.S. Pat. No. 4,948,631 discloses a process for preparing these bluish pearl luster pigments.

Additionally, JP-A-164 653/1983, JP-A-126 468/1984, JP-A-184 570/1985 and U.S. Pat. Nos. 6,139,614 and 4,623,396 disclose reduced titanium oxide coated mica.

U.S. Pat. No. 5,624,487 teaches that very bluish clean-hued pigments are obtained only by reducing the costly, blue-reflecting micas having a high TiO₂ layer thickness. The less costly silvery micas having a low TiO₂ layer thickness produces blues having a high green content. U.S. Pat. No. 5,624,487 teaches to overcome this problem by treatment of the thin layered titania coated mica with a vaporized organic compound and ammonia at temperatures around 800 to 900 C.

U.S. Pat. No. 7,014,700 discloses self supporting platy TiO₂ reduced at elevated temperatures of about 675-850 C in a reducing atmosphere.

Wang Y. et al., J. Crystal Growth 282, (2005) 402-406 discloses the preparation of blue titanium suboxide nanoparticles (average grain size of less than 70 nm) from metallic titanium by arc plasma techniques in the presence of Ar, H₂ and H₂O. The particles of Wang Y. contain a core of titanium. The particles of the present invention preferably does not contain a core of titanium.

Hauf C. et al., J. of Materials Science 34, (1999) 1287-1292 discusses the preparation of titanium suboxides powders by reduction of TiO₂ powders with silicon. In addition, the reduction of TiO₂ coated mica pigments with silicon is described. The particles of Hauf C. contain silicon oxide.

WO2005118480 is directed to a method of continuously producing a pre-determined suboxide, the method comprising continuously feeding an oxide starting material, such as titanium dioxide, into a reaction chamber and contacting the so-fed oxide with a substantially moisture-free gas (hydrogen, carbon, carbon monoxide, methane, propane or other hydrocarbons) and collecting the pre-determined suboxide (titanium suboxide).

The presently most preferred reduced TiO₂ particles are however produced using a plasma torch. The plasma torch is preferably an induction plasma torch. The preferred induction plasma torches for use in the present invention are available from Tekna Plasma Systems, Inc. of Sherbrooke, Quebec, Canada. Boulos et al., U.S. Pat. No. 5,200,595, is hereby incorporated by reference for its teachings relative to the construction and operation of plasma induction torches.

WO06/131472 (U.S. application Ser. No. 11/446,849) discusses a process for treating coated platelet-like substrates in a plasma stream and is herein incorporated entirely by reference.

The present inventors have discovered that this pigment combination gives particular advantages. Combining the bluish, blue/black hued titanium suboxide(s), titanium oxynitrides or titanium nitride particles with a luster pigment by mixing or deposition gives highly reproducible color effect. For example, reduction of TiO₂ coated mica to form suboxides, the resulting coloration depends very much on the coating thickness, purity of the TiO₂ and uniformity of the coating. Furthermore, when the TiO₂ mica is placed in a reducing atmosphere, normally at high temperatures (around 860 C and above), the substrate (mica, glass etc.) becomes more fragile and thus the integrity of the flake is at risk and the luster effect is lost when added to paints, cosmetics, plastics and coatings.

The present pigment combination overcomes these disadvantages by reducing TiO₂ or titanium suboxide precursor particle per se, then combining with the luster pigment or flake. In this way, only the titanium suboxide (or suboxynitride) precursor is subjected to the high temperatures. The make-up and purity of the reduced particles may be varied allowing for great flexibility in the resulting color of the reduced TiO₂ or directly formed suboxide species. The suboxide particle may then be deposited, coated onto the component B particle or simply mixed with component B. The coating or deposition may be applied to many different platelet-like particles under mild conditions which allows retention of the platelet-like particle shape of component B. Therefore, color effect is intensified, and new color effects may be achieved.

Accordingly, the present invention is directed to a pigment mixture comprising two different particulate components A and B, wherein component A is a reduced titanium oxide particle, comprising titanium suboxide(s), titanium oxynitride and/or titanium nitride, and component B particle is an organic, or inorganic pigment.

In addition, the present invention encompasses compositions comprising a pigment mixture comprising two different particulate components A and B, wherein component A is a reduced titanium oxide particle, comprising titanium suboxide(s), titanium oxynitride and/or titanium nitride and component B is an organic, or inorganic pigment.

The component A particle is preferably a spherical particle, comprising at least a titanium suboxide(s), a titanium oxynitride or a titanium nitride a species or mixture of species thereof.

Examples of component B are mica, glass, SiO₂, BiOCl, graphite or mixtures thereof. Especially suitable as component B are effect pigments, such as pearlescent pigments, or metal effect pigments.

The invention likewise provides for coatings such as paints and varnishes, printing inks, plastics, such as films and molded bodies, masterbatches, agricultural films, personal care products and cosmetic comprising the pigment mixture of the invention.

The invention also encompasses a method of forming a (multilayer) structure comprising the steps of:

reducing TiO₂ particles to form a component A particle, and

applying or coating said component A particle onto a platelet-like component B, which is different than Component A.

In said method Component A is preferably spherical and Component B is platelet-like and the diameter ratio of Component A:Component B is at least 1:2.

Component A particle comprises depending on the reduction method titanium suboxide(s), titanium oxynitride, titanium nitride species or mixtures of species thereof.

In a preferred embodiment, the component A particle composition may also contain unreduced TiO₂.

Titanium suboxide(s) for purposes of the invention means TiO₂ has been wholly or partially converted to reduced titanium species (oxidation state of the titanium: from <4 to 2). The sequence is TiₙO₂ₙ₋₁ (1≦n≦9). For example, Ti₂O₃, Ti₃O₅, Ti₄O₇, T₅O₉, Ti₉O₁₇ and TiO are TiO_{suboxides}.

The titanium suboxide will normally be characterized by a bluish coloration even when some TiO₂ remains unreduced in the particle. That is, the pigment mixture of the present invention has a bluish hue shift, wherein the hue difference (delta b) is expressed as a CIELAB quantity and is negative. The addition of TiOₓ particles to component B causes a decrease in b*. A compacted opaque powder in air features a bluish hue which is expressed as a CIELAB quantity b*≦−1, especially b*≦−5, measured at an angle difference of 20° to the gloss angle, standard illuminant D65. Thus more negative b* values may be preferred depending on the application and the properties of Component B. For example, b* values of −15, ≦−20 and below are possible. The darkness of the blue is measured by the factor L (lightness). So it is possible to have very bright blues with a very negative b*.

TiO₂ may be reduced entirely or partially to form TiN depending on the reduction conditions. A large amount of TiN in component A particle will give the particle a bronze or golden color.

It is important to understand that when a TiO₂ particle is reduced the particle produced (component A) may be any combination of various titanium suboxides, titanium oxynitrides, titanium nitride and unreduced TiO₂. It is also understood that component A excludes such particles as those defined by component B.

Component A particles may contain various concentrations of titanium suboxide, titanium oxynitrides, titanium nitride and unreduced TiO₂ species The various species may form a particle which exhibits a random arrangement of those reduced species, or a particular arrangement from the surface to the core of the particle. For example, the most reduced species may be distributed closer to the surface and the greatest concentration of unreduced TiO₂ may be present at the core.

Component A particle may be virtually any shape such as for example, spherical, rod-like, flake or platelet-shaped. It is preferred that the shape is spherical or rod-like. Spherical component A particles are most preferred and are new.

Accordingly, the present invention relates also to spherical particles, comprising at least a titanium suboxide, a titanium oxynitride or a titanium nitride a species or mixture of species thereof.

The titanium suboxide species is defined as wholly or partially reduced TiO₂with an oxidation state of the titanium: from <4 to 2.

The particle size of the titanium suboxide particles range from about 30 nm to about 30 μm, preferably about 30 nm to about 10000 nm or most preferably 30 nm to about 5000 nm, especially about 30 nm to about 500 nm.

Preferably, the particles further contain unreduced TiO₂.

The process for producing the particles, comprising at least a titanium suboxide, a titanium oxynitride or a titanium nitride a species or mixture of species thereof, comprises treating TiO₂ particles using a plasma torch.

The particles, especially a spherical, rod-like, flake or platelet-shaped particles, obtainable by the process are new and form a further object of the present invention.

The component A particle comprising titanium suboxide, a titanium oxynitride or a titanium nitride is self-supporting. By self-supporting, it is meant that the titanium suboxide, titanium oxynitride and/or a titanium nitride particle is not derived from a titanium dioxide coated substrate particle, such as titanium dioxide coated mica, but is preferably produced from titanium dioxide particles.

For example, U.S. Pat. No. 5,611,851 discloses substantially substrate free platy TiO₂. Titanium dioxide coated mica is treated with mineral acid and phosphoric acid followed by an extractive dissolution using an alkali. A platy substantially substrate-free TiO₂ is produced. The platy TiO₂ of U.S. Pat. No. 5,611,851 incompletely removes the substrate, leaving a product with at least some mica.

The particle sizes for component A may range from about 30 nm, especially 60 nm to about 30 μm. For example, about 30 nm to about 10000 nm or even 30 nm, especially 100 nm to about 5000 nm, or 10 nm to 100 nm are envisioned. For example about 30 nm to about 500 nm are possible.

Preferred starting materials are non-treated anastase, or rutile TiO₂ particles with an equivalent spherical diameter<10 μm, especially <5 μm, very especially <1 μm, which are commercially available, for example, from DuPont, Kronos, Sachleben Chemie, Millenium Chemicals, and Huntsman Tioxide. Examples of such materials are Kronos® 1001 (anastase TiO₂), Kronos® 2900 (rutile TiO₂).

An especially preferred technique for generating component A is reduction of TiO₂ in a plasma chamber.

The plasma torch is preferably an induction plasma torch but might be a arc plasma. The preferred induction plasma torches for use in the process of the present invention are available from Tekna Plasma Systems, Inc. of Sherbrooke, Quebec, Canada.

The induction plasma torch used to treat component particle A is equipped with a powder feeder that operates by entraining the particles in an, upward or downward, stream of gas for transport to the plasma induction torch. In addition, it is also possible to inject the particles as a slurry (e.g. aqueous, organic solvents, such as C₁₋₄alcohols, ketones, and di-C₁₋₂alkyl ethers) into the plasma reactor. This slurry is atomized at the tip of the injection probe.

In a preferred embodiment of the invention the transport gas is inert, i.e. does not react with the outer surfaces of the particles. Typically, the fluidizing gaseous medium is selected to be compatible with the particles, i.e. do not substantially adversely affect the quality of the particles. Examples of such transport gases are argon, nitrogen, helium, or mixtures such as argon/hydrogen and argon/oxygen. Generally, gases such as nitrogen, argon, helium and the like, can be used, where no substantial adverse oxidation reaction of the particles takes place. Gases used to modify the outer surface of the particles are for example, reducing gases such as ammonia, hydrogen, methane, carbon monoxide, other hydrocarbons such as methane, propane and butane and mixtures thereof. In a preferred embodiment of the present invention the transport gas is argon and the gas used to modify the outer surface of the particles is hydrogen.

Reduced TiO₂ particles may be partially or wholly converted to reduced titanium species (oxidation state of the titanium: from <4 to 2), titanium oxynitride and titanium nitride.

For example, titanium dioxide particles may be treated with any of the above reducing gases, in a nonoxidizing atmosphere in the plasma chamber thus partially or wholly converting titanium dioxide species to titanium suboxides, titanium oxynitride, titanium nitride species.

Spherical particles, comprising at least a titanium suboxide, a titanium oxynitride or a titanium nitride a species or mixture of species thereof, obtained by the above described process are new and form a further subject of the present invention.

It may also be possible to achieve suboxides without a reducing gas. For example, injection of TiO₂ in a plasma in 10% argon in air may also achieve suboxides due to the lability of the oxide sublattice at elevated temperatures. Oxide vacancies are created at high temperatures if the partial pressure of oxygen is low.

Within the meaning of the present invention spherical particles are defined as particles having an aspect ratio of less than 10, especially of less than 2, very especially 1.

The transport gas in the plasma reactor is, for example, a mixture of about 80-90% argon, with the balance being hydrogen. By using said mixture of argon and hydrogen as transport gas it is, for example, possible to convert titanium dioxide, at least partially, to a titanium suboxide. Accordingly, in a preferred embodiment the present invention is directed to particles consisting of titanium suboxides and optionally TiO₂, wherein the content of the TiO₂ is highest in the core of the particles and decreases to the surface of the particles.

Another example of a reactive transport gas is ammonia. By using ammonia titanium nitride/oxy nitride particles may be produced.

There are several points in the formation of component A where the gases may be introduced into the plasma chamber in order to achieve a particular effect on the surface of the particle.

For example, the gas or gases may be introduced in the carrier, dispersion or quench gas streams.

During this process, because the outer surfaces of the component A particles are melted while entrained in a gas, the obtained particles may have a smooth outer surface. After melting, the particles fall through a distance sufficient to permit cooling and at least partial solidification prior to contact with a solid surface or each other.

This smooth surface may be preferred in cosmetic formulations. A spherical surface may also be preferable. Among other things, the smoothing of the particle surface may suppress pores on the surface of the particle and may help to avoid agglomeration of component A particles.

The actual chemical functionality, such as hydroxyl groups at the surface of the treated particle may also be altered when the component A particle is treated in a plasma chamber thus improving stability to light and other environmental factor.

If TiO₂ is processed by an induction plasma torch to form the titanium suboxide, titanium oxynitride, titanium nitride, unreduced TiO₂ or mixtures thereof, the resulting composition may be amorphous or crystalline. For example, the mixture of unreduced TiO₂ may be rutile or anatase and contain reduced species of titanium suboxide such as Ti₄O₇ of a different crystallinity. It is also possible for the reduced species to maintain some rutile character. Such reduced species of Ti₄O₇ may improve the photostability of the mixture. The starting TiO₂ may be anatase or rutile and the reduction by induction plasma may transform anatase to rutile and/or transform rutile to anatase.

A titanium suboxide species may be especially useful in applications which require high photostability or applications such as cosmetics which may require no tin oxide component.

Component B particle is an organic, or inorganic pigment.

In a preferred embodiment of the present invention the pigment is an effect pigment, i.e. a pigment which imparts besides color additional properties to the application medium, such as, for example, angle dependent color change (flop), luster, or texture. Examples of effect pigments are pearlescent (nacreous) pigments, metal effect pigments, interference pigments and luster pigments.

Component B particle is virtually any (multilayer) structure leading to interference colors, such as special-effect pigments, luster or nacreous pigment. Particle B may also comprise non-multilayer structures such as an organic pigment, inorganic pigment, mica, a metal or metal oxide, glass, SiO₂, BiOCl, graphite or mixtures thereof.

It is understood that component B is different than component A.

Multilayered structures leading to interference colors are often referred to as special-effect pigments, luster or nacreous pigments and well known in the art and commercially available under such tradenames as Xymara® available from Ciba Chemicals Inc.

In principle component B might comprise all platelet-like effect pigments, such as, for example, platelet-like iron oxide, bismuth oxychloride or platelet-like materials coated with colored or colorless metal oxides, such as, for example, natural or synthetic micas, other laminated silicates such as talc, kaolin or sericite or glass platelets can be used. Mica flakes coated with metal oxides such as are disclosed, for example, in U.S. Pat. Nos. 3,087,828 and 3,087,829 are particularly preferred as substrates, herein entirely incorporated by reference. Metal oxides are both colorless, highly refractive metal oxides, such as, in particular, titanium dioxide and/or zirconium dioxide, as well as colored metal oxides, such as, for example, chromium oxide, nickel oxide, copper oxide, cobalt oxide and in particular iron oxides, such as, for example, Fe₂O₃, or Fe₃O₄, or mixtures of such metal oxides. Such metal oxide/mica pigments are commercially available under the tradenames Afflair® and Iriodin®. According to EP-A-373575 these substrates are coated with an optionally hydrated silica layer or with a layer of another insoluble silicate such as, for example, aluminum silicate.

These (multilayer) structures frequently are formed from a core of natural micaceous iron oxide (for example as in WO99/48634), synthetic and doped micaceous iron oxide (for example as in EP-A-068311), mica (muscovite, phlogopite, fluorophlogopite, synthetic fluorophlogopite, talc, kaolin), basic lead carbonate, flaky barium sulfate, SiO₂, Al₂O₃, TiO₂, glass, ZnO, ZrO₂, SnO₂, BiOCl, chromium oxide, BN, MgO flakes, Si₃N₄, graphite, pearlescent pigments (including those which react under the fluidized bed conditions to nitrides, oxynitrides or by reduction to suboxides etc.) (for example EP-A-0948571, U.S. Pat. Nos. 6,773,499, 6,508,876, 5,702,519, 5,858,078, WO98/53012, WO97/43348, U.S. Pat. No. 6,165,260, DE-A-1519116, WO97/46624, EP-A-0509352), pearlescent (multilayer) pigments (for example EP-A-0948572, EP-A-0882099, U.S. Pat. Nos. 5,958,125, 6,139,613), coated or uncoated SiO₂ spheres (for example known from EP-A-0803550, EP-A-1063265, JP-A-11322324), EP-A-0803550, EP-A-1063265, JP-A-11322324). Particularly preferred cores are mica, SiO₂ flakes, Al₂O₃ flakes, TiO₂ flakes, Fe₂O₃ flakes, BiOCl and glass flakes.

The glass flake cores for the purpose of the invention include any of the known grades such as A-glass, E-glass (high resistivity makes E-glass suitable for electrical lami nates), C-glass and ECR-glass (corrosion grade glass) materials.

For example, component B particle may be a platelet-like (multilayered) structures such as:

| | | | |
|---|---|---|---|
| TRASUB | TiO₂ | | |
| TRASUB | TiO₂ | Fe₂O₃ | |
| TRASUB | TiO₂ | Fe₃O₄ | |
| TRASUB | titanium suboxide(s) | | |
| TRASUB | TiO₂ | TiN | |
| TRASUB | TiO₂ | SiO₂ | |
| TRASUB | TiO₂ | titanium | |
| | | suboxide(s) | |
| TRASUB | TiO₂ | TiON | TiN |
| TRASUB | TiO₂ | SiO₂ | TiO₂ |
| TRASUB | TiO₂ | SiO₂ | silicon suboxide(s) |
| TRASUB | TiO₂ | SiO₂ | Fe₂O₃ |
| TRASUB | TiO₂ | SiO₂ | TiO₂/Fe₂O₃ |
| TRASUB | TiO₂ | SiO₂ | (Sn,Sb)O₂ |
| TRASUB | SnO₂ | TiO₂ | |
| TRASUB | SnO₂ | TiO₂ | Fe₂O₃ |
| TRASUB | (Sn,Sb)O₂ | SiO₂ | TiO₂ |
| TRASUB | Fe₂O₃ | SiO₂ | (Sn,Sb)O₂ |
| TRASUB | TiO₂/Fe₂O₃ | SiO₂ | TiO₂/Fe₂O₃ |
| TRASUB | Cr₂O₃ | SiO₂ | TiO₂ |
| TRASUB | Fe₂O₃ | | |
| TRASUB | Fe₂O₃ | SiO₂ | TiO₂ |
| TRASUB | titanium suboxide(s) | SiO₂ | titanium suboxide(s) |
| TRASUB | TiO₂ | SiO₂ | TiO₂ + SiO₂ + TiO₂ |
| TRASUB | TiO₂ + SiO₂ + TiO₂ | SiO₂ | TiO₂ + SiO₂ + TiO₂ |
| TRASUB | TiO₂ | Al₂O₃ | TiO₂ |
| TRASUB | Fe₂TiO₅ | SiO₂ | TiO₂ |
| TRASUB | TiO₂ | SiO₂ | Fe₂TiO₅/TiO₂ |
| TRASUB | TiO₂ | SiO₂ | MoS₂ |
| TRASUB | TiO₂ | SiO₂ | Cr₂O₃ |
| TRASUB | TiO₂ | SiO₂ | TiO₂ + SiO₂ + TiO₂ + |
| | | | Prussian Blue |
| TRASUB | TiO₂ | STL | |

wherein TRASUB is a semitransparent, or transparent substrate having a low index of refraction selected from the group consisting of natural, or synthetic mica, another layered silicate, glass, Al₂O₃, SiO_{z}, SiO₂, SiO₂/SiOₓ/SiO₂ (0.03≦x≦0.95), SiO_{1.40-2.0}/SiO_{0.70-0.99}/SiO_{1.40-2.0}, or Si/SiO_{z} with 0.70 z 2.0, and

STL is a semi-transparent layer selected from the group consisting of a semi-transparent metal layer of Cu, Ag, Cr, or Sn, or a semi-transparent silicon suboxide(s), titanium suboxide(s) or carbon layer.

The (multilayered) pigments above may also include an absorption pigment as an additional layer. For example a further coating with Prussian blue or red-carmine on an interference pigment allows for striking color effects.

Pigments based on TiO₂ coated, platelet-like, transparent substrates are preferred, wherein those are most preferred, wherein the thickness of the TiO₂ layer results in a silver-like color.

Other layered structures envisioned for component B are:

(a) a transparent substrate having a low index of refraction selected from the group consisting of natural, or synthetic mica, another layered silicate, glass, Al₂O₃, SiO₂, SiO₂, SiO₂/SiOₓ/SiO₂ (0.03≦x≦0.95), SiO_{1.40-2.0}/SiO_{0.70-0.99}/SiO_{1.40-2.0}, or Si/SiO_{z} with 0.70 z 2.0 and

(b) a layer of a metal oxide of high refractive index on the substrate selected from the group consisting of ZrO₂, Fe₂O₃, or TiO₂; or

(a) a transparent substrate having a low index of refraction selected from the group consisting of natural, or synthetic mica, another layered silicate, glass, Al₂O₃, SiO_{z}, especially SiO₂, SiO₂/SiOₓ/SiO₂ (0.03≦x≦0.95), SiO_{1.40-2.0}/SiO_{0.70-0.99}/SiO_{1.40-2.0}, or Si/SiO_{z} with 0.70 z 2.0, and

(b) a reflecting layer or a semitransparent layer, or a semitransparent metal layer; or

(a) a platelet shaped titanium dioxide substrate,

(b) a layer of Fe₂O₃, Fe₃O₄, FeOOH, Cr₂O₃, CuO, Ce₂O₃, Al₂O₃, SiO₂, BiVO₄, NiTiO₃, CoTiO₃ and also antimony-doped, fluorine-doped or indium-doped tin oxide; or

(a) a platelet shaped iron oxide substrate,

b) a colorless coating having a refractive index n≦1.8, and

c) a colorless coating having a refractive index n≧2.0.

Alternative (multilayer) structures for component B might comprise flakes comprising layers (a), (b) and optionally (c):

(a) a metallic platelet-shaped substrate selected from the group consisting of titanium, silver, aluminum, copper, chromium, iron, germanium, molybdenum, tantalum, or nickel, and

(b) a layer of a metal oxide of low refractive index or of high refractive index on the substrate

(c) an optional layer comprising a semi-transparent metal oxide selected from the group consisting of SiO_{z}, SiO₂/SiO_{z}, titanium suboxide(s), TiO₂/titanium suboxide(s) and 0.70≦z≦2.0.

Moreover, flakes comprising layered structures of (a), (b) and (c) below are preferred:

(a) a transparent substrate having a low index of refraction selected from the group consisting of natural, or synthetic mica, another layered silicate, glass, Al₂O₃, SiO_{z}, SiO₂, SiO₂/SiOₓ/SiO₂ (0.03≦x≦0.95), SiO_{1.40-2.0}/SiO_{0.70-0.99}/SiO_{1.40-2.0}, or Si/SiO_{z}with 0.70≦z≦2.0, and

(b) a titanium dioxide layer,

(c) a layer of hydrous aluminum oxide, a layer of hydrated zirconium oxide, a top layer comprising hydrated zirconium oxide obtained by hydrolysis in the presence of a hypophosphite, and a hydrated metal oxide, or a combination of hydrated cerium and aluminum oxides, or a layer (topcoat) which contains a polysiloxane and a rare earth metal compound.

Furthermore platelet-like particles, comprising

(a) a core and

(b) a polymeric coating, comprising nitrogen and carbon atoms, on the surface of the flakes are envisioned.

Additionally, platelet-like particles, comprising

(a) a substrate, and

(b) a layer of a metal nitride/oxy nitride, titanium suboxide(s), SiO_{z} or SiO₂/SiO_{z}, wherein 0.70≦z≦2.0 are possible as component B.

The (multilayered) structures may be spherical, rod-like or platelet-shaped substrates. Platelet, flakey shapes are preferred.

The component B may also be an organic color pigment or a conventional inorganic pigment.

Suitable colored pigments especially include organic pigments selected from the group consisting of azo, azomethine, methine, anthraquinone, phthalocyanine, perinone, perylene, diketopyrrolopyrrole, thioindigo, dioxazine iminoisoindoline, dioxazine, iminoisoindolinone, quinacridone, flavanthrone, indanthrone, anthrapyrimidine and quinophthalone pigments, or a mixture or solid solution thereof; especially a dioxazine, diketopyrrolopyrrole, quinacridone, phthalocyanine, indanthrone or iminoisoindolinone pigment, or a mixture or solid solution thereof.

Colored organic pigments of particular interest include C.I. Pigment Red 202, C.I. Pigment Red 122, C.I. Pigment Red 179, C.I. Pigment Red 170, C.I. Pigment Red 144, C.I. Pigment Red 177, C.I. Pigment Red 254, C.I. Pigment Red 255, C.I. Pigment Red 264, C.I. Pigment Brown 23, C.I. Pigment Yellow 109, C.I. Pigment Yellow 110, C.I. Pigment Yellow 147, C.I. Pigment Orange 61, C.I. Pigment Orange 71, C.I. Pigment Orange 73, C.I. Pigment Orange 48, C.I. Pigment Orange 49, C.I. Pigment Blue 15, C.I. Pigment Blue 60, C.I. Pigment Violet 23, C.I. Pigment Violet 37, C.I. Pigment Violet 19, C.I. Pigment Green 7, C.I. Pigment Green 36, or a mixture or solid solution thereof.

Suitable colored pigments also include inorganic pigments, including phosphorescent and fluorescent inorganic pigments; especially those selected from the group consisting of metal oxides, antimony yellow, lead chromate, lead chromate sulfate, lead molybdate, ultramarine blue, cobalt blue, manganese blue, chrome oxide green, hydrated chrome oxide green, cobalt green and metal sulfides, such as cerium or cadmium sulfide, cadmium sulfoselenides, zinc ferrite, bismuth vanadate, Prussian blue, Fe₃O₄, carbon black and mixed metal oxides.

It is preferred that component B particle is plate-like or alternatively described as flakes or parallel structures. Generally the flakes have a length of from 1 μm to 5 mm, a width of from 1 μm to 5 mm, and a thickness of from 20 nm to 2 μm, and a ratio of length to thickness of at least 2:1, the particles having two substantially parallel faces, the distance between which is the shortest axis of the core.

The component B flakes of the present invention are not of a uniform shape. Nevertheless, for purposes of brevity, the flakes will be referred to as having a “diameter”. The flakes have a thickness of from 20 to 2000 nm, especially from 50 to 1000 nm. It is presently preferred that the diameter of the flakes be in a preferred range of about 1-60 μm with a more preferred range of about 5-40 μm.

Preferred component B particles are any high aspect ratio materials, such as platelets (flakes), rod-like materials and fibers. The aspect ratio is at least 10 to 1. The term “aspect ratio” refers to the ratio of the maximum to the minimum dimension of a particle.

Thus, the aspect ratio of the flakes of the present invention is in a preferred range of about 2.5 to 625.

The two components A and B may be mixed to form a pigment composition (physical mixture).

Alternatively, component(s) A may be coated or deposited onto component B.

A pigment mixture (composite pigment) may also be prepared by a process comprising spray-drying an aqueous suspension consisting of discrete particles of component A and B (cf. U.S. Pat. No. 5,562,763).

The component A may be added to a slurry of a (multilayered) platelet-like such as a pearlescent mica based pigment (component B) in an organic solvent such as ethylene glycol or isopropanol, and then the slurry is treated with ultrasound for 5 minutes to 2 hours, filtered and dried. The final coated flake may be calcined and further coated with for example a protective layer such as SiO₂. U.S. Pat. No. 5,407,746 discloses TiO₂ coatings onto a plate-like substrate using a similar process as above. Component A may be trapped in a metal oxide layer, such as an aluminum oxide layer, additionally precipitated on component B. The process for the preparation of such particles involves the precipitation of the metal oxide onto component B in the presence of component A.

A pigment mixture can also be prepared as described in U.S. Pat. No. 4,772,331 by admixing a suspension or solution of component A and a high molecular weight organic compound in water and/or an alcohol with a suspension of component B in a solution of a high molecular weight organic compound in water and/or alcohol whereby said component B is precipitated onto the surface of said component B and bound thereto by said high molecular weight organic compound.

Alternatively, component B with a surface modified by component A may be obtained as described in WO2005/056696 by trapping component A in one or several layers of immobilised LOST and/or UCST polymers.

The LOST (LOST=lower critical solution temperature) polymers may be selected from polyalkylenoxide derivatives, olefinic modified PEO-PPO-co-polymers, polymethylvinylether, poly-N- vinylcaprolactam, ethyl-(hydroxyethyl)-cellulose, poly-(N-isopropylacrylamide) and polysiloxanes and mixtures thereof.

The UCST (UCST=Upper Critical Solution Temperature) polymers may be selected from polystyrol, polystyrol-copolymers and polyethylenoxide-copolymers and mixtures thereof. The substrate is contacted with a solution from a polymer that has UCST properties and a layer is deposited on the surface of the substrate by reducing the temperature of the polymer solution (WO03014229).

When component A titanium suboxide particles are spherical and allowed to coat an interference pigment such as a mica/TiO₂ plate-like particle a (multilayered) structure is formed with improved softness. This improved property is especially important when the plate-like particle is used in cosmetic applications for skin. Thus the coating with the spherical titanium suboxide particles not only affects the color characteristics of the formed (multilayered) structure but also affects the feel of the (multilayered) structure on the skin.

In order to get the softening effect, the diameter of the component A is smaller than the diameter of component B. For example, the diameter ratio of A:B will vary from about 1:1000 to about 1:2 or the diameter ratios of A:B must be at least 1:2. Preferably the diameter ratio of A:B may vary from about 1:100 to about 1:5.

Alternatively, the component A may be dispersed in a polymeric organic binder to form a liquid. The liquid may be used to coat or deposit onto the component B to form the mixed pigment. The pigment may then be calcined at temperatures high enough to burn off the polymeric organic binder. The mixed pigment now comprises the original core (multilayer) structure encapsulated by an additional layer of component A.

The weight ratio of component A to component B may be any ratio. For example the ratio may be about 1:10 to about 10:1, alternatively about 5:1 to about 1:5.

The wt. % A component may vary from about 10 to about 50 wt. % component B. For example, if component B is 100 grams, component A may be about 20 grams or 20 wt. % of the dry weight of component B.

This weight ratio may depend upon the diameter of the A component in relation to the diameter of component B. Thus if diameter A component is small in relation to diameter of component B, a smaller wt. % of component A may be effective. For example, less than 30 wt. % component A to about 100 wt. % B are possible.

The “diameter” of the B component flakes are for example, at least twice the size of the component A sphere diameter.

The pigment mixtures of the invention may be incorporated in coatings, ceramics, glasses plastics, films, agricultural films, button pastes, masterbatches, seed coatings, printing inks, cosmetics and personal care products. Accordingly, the present invention relates to coatings, varnishes, plastics, paints, printing inks, masterbatches, ceramics or glasses, cosmetics or personal care products, comprising the particles of the present invention, or the pigment mixture of the present invention.

The concentration of the pigment mixture in the system in which it is to be used for pigmenting is generally between 0.01 and 50% by weight, preferably between 0.1 and 5% by weight, based on the overall solids content of the system. This concentration is generally dependent on the specific application.

Plastics comprising the pigment mixture of the invention in amounts of from 0.1 to 50% by weight, in particular from 0.5 to 7% by weight, are frequently notable for a bluish/grey or bluish/black lustrous metallic effect.

In the coating sector, especially in automotive finishing, the pigment mixture is employed in amounts of 0.5-10% by weight. The proportion in which the component A are mixed with component B, depends on the desired effect.

The invention likewise provides pigment preparations comprising components A and B with binders and, if desired, additives, the said preparations being in the form of substantially solvent-free, free-flowing granules. Such granules contain up to 95% by weight of the pigment mixture. A pigment preparation in which the pigment mixture of the invention is pasted up with a binder and with water or an organic solvent, with or without additives, and the paste is subsequently dried and brought into a compact particulate form, e.g. granules, pellets, briquettes, a masterbatch or tablets.

The mixtures are highly suitable for coloring plastics or high molecular weight materials which can be further processed to fibers, cast and molded articles, films or coating compositions such as solvent or water based coatings, which are for example conventionally employed in the automobile industry.

Thus, the high molecular weight organic material may be an industrial paint, automotive paint, molded article or film.

Suitable high molecular weight organic materials include thermoplastics, thermoset plastics or elastomers, natural resins or casein for example, cellulose ethers; cellulose esters such as ethyl cellulose; linear or crosslinked polyurethanes; linear, crosslinked or unsaturated polyesters; polycarbonates; polyolefins such as polyethylene, polypropylene, polybutylene or poly-4-methylpent-1-ene; polystyrene; polysulfones; polyamides; polycycloamides; polyimides; polyethers; polyether ketones such as polyphenylene oxides; and also poly-p-xylene; polyvinyl halides such as polyvinyl chloride, polyvinylidene chloride, polyvinylidene fluoride or polytetrafluoroethylene; acrylic polymers such as polyacrylates, polymethacrylates or polyacrylonitrile; rubber; silicone polymers; phenol/formaldehyde resins; melamine/formaldehyde resins; urea/formaldehyde resins; epoxy resins; styrene butadiene rubber; acrylonitrile-butadiene rubber or chloroprene rubber; singly or in mixtures.

High molecular weight for purposes of the invention means an average molecular weight of from about 10² to about 10⁶ g/mole.

In particular the component A and B mixtures may be incorporated into skin-care products, bath and shower additives, preparations containing fragrances and odoriferous substances, hair-care products, deodorizing and antiperspirant preparations, decorative preparations, light protection formulations and preparations containing active ingredients and uses thereof to achieve special color effects.

Body-care products are, in particular, skin-care products, such as body oils, body lotions, body gels, treatment creams, skin protection ointments, shaving preparations, such as shaving foams or gels, skin powders, such as baby powder, moisturizing gels, moisturizing sprays, revitalizing body sprays, cellulite gels and peeling preparations.

In a preferred embodiment of the present invention the personal care product is a body-care product for the skin and its adnexa.

Suitable bath and shower additives are shower gels, bath-salts, bubble baths and soaps.

Preparations containing fragrances and odoriferous substances are in particular scents, perfumes, toilet waters and shaving lotions (aftershave preparations).

Suitable hair-care products are, for example, shampoos for humans and animals, in particular dogs, hair conditioners, products for styling and treating hair, perming agents, hair sprays and lacquers, hair gels, hair fixatives and hair dyeing or bleaching agents.

Suitable decorative preparations are in particular lipsticks, nail varnishes, eye shadows, mascaras, dry and moist make-up, rouge, powders, depilatory agents and suntan lotions.

Suitable cosmetic formulations containing active ingredients are in particular hormone preparations, vitamin preparations and vegetable extract preparations.

The mentioned body-care products may be in the form of creams, ointments, pastes, foams, gels, lotions, powders, make-ups, sprays, sticks or aerosols.

The present invention therefore also relates to a body-care product comprising components A and B.

The mixture of components A and B are present in the body care and household products in a concentration of about 0.0001% to about 25%, based on the total formulation, preferably from about 0.001% to about 15%, and most preferably from about 0.05% to about 10%.

The present pigment mixtures are particularly suitable for coloration of cosmetic and body care products, in particular:
skin-care preparations, e.g. skin-washing and cleansing preparations in the form of tablet-form or liquid soaps, soapless detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils; body oils, body lotions, body gels; skin protection ointments;
cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care preparations, such as nail varnish, nail varnish removers, nail hardeners or cuticle removers;
foot-care preparations, e.g. foot baths, foot powders, foot creams or foot balsams, special deodorants and antiperspirants or callus-removing preparations;
light-protective preparations, such as sun milks, lotions, creams or oils, sunblocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances and odoriferous substances containing preparations (scents, eau de Cologne, eau de toilette, eau de parfum, parfum de toilette, perfume), perfume oils or perfume creams;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colourants, preparations containing self-oxidising dyes, or natural hair colourants, such as henna or camomile;
decorative preparations, in particular lipsticks, nail varnishes, eye shadows, mascaras, dry and moist make-up, rouge, powders, depilatory agents and suntan lotions
cosmetic formulations containing active ingredients, in particular hormone preparations, vitamin preparations, vegetable extract preparations and antibacterial preparations.

### Presentation Forms

The final formulations containing the pigment mixtures may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a stick,
in the form of a spray (spray with propellent gas or pump-action spray) or an aerosol,
in the form of a foam, or
in the form of a paste.

Examples of body care products of the present invention are listed in the Table below:

| Body care product | Ingredients |
|---|---|
| moisturizing cream | vegetable oil, emulsifier, thickener, perfume, water, |
| | stabilizers, preservatives, dyes/pigments |
| Shampoo | surfactant, emulsifier, preservatives, perfume, |
| | antioxidant, UV absorbers, dyes/pigments |
| Lipstick | vegetable oils, waxes, stabilizers, dyes/pigments |
| eye shadow | Talc, Zinc Stearate, oils, stabilizers, pigments |
| Makeup | Water, thickener, oils, emulsifier, perfume, |
| | preservatives, stabilizers, pigments |

Various features and aspects of the present invention are illustrated further in the examples that follow. While these examples are presented to show one skilled in the art how to operate within the scope of this invention, they are not to serve as a limitation on the scope of the invention where such scope is only defined in the claims. Unless otherwise indicated in the following examples and elsewhere in the specification and claims, all parts and percentages are by weight, temperatures are in degrees centigrade and pressures are at or near atmospheric.

### EXAMPLES

### Example 1

Titanium dioxide powder is fluidized in a stream of argon and fed at a rate of 10 kg/hour into a a plasma reactor with a Tekna PL-70 RF induction plasma torch operated at an oscillator frequency of 3 MHz and a power of 90 KW. The sheath gas is a mixture of 150 slpm argon and 10 slpm hydrogen and the central gas is argon at 40 slpm. The operating pressure is maintained at slightly lower than atmospheric pressure.

The Powder X-Ray Diffraction results:

a) A feedstock of anatase Ti0₂ (Kronos® 1001) resulted in a mixture of 20 to 30% Ti₄O₇, 10% rutile and >50% anatase TiO₂.

b) A feedstock of rutile TiO₂ (Kronos® 2900) resulted in a mixture of anatase and rutile TiO₂ and >50% Ti₄O₇.

### Measurement of the Color of 0.38 mm Rolled PVC Sheets:

### Premix:

Titanium suboxide pigment (0.4 g (or 0.2 g) is mixed for 30 minutes with 14.0 g of a base mixture and then slowly stirred with 26.0 g of polyvinyl chloride (PVC) (EVIPOL® SH 7020, EVC GmbH). The base mixture consists of plastiziser (12.9 g Palatinol® 10P (di-2-propylheptylphthalate, BASF), 0.6 g Drapex® 39 (epoxidised soya bean oil, Witco Vinyl Additives GmbH) and 0.5 g Mark BZ 561 (Crompton Vinyl Additives GmbH).

### Production of Rolled Sheets:

The mixture of PVC and pigment/base mixture obtained above is rolled in a 2-roll mill (Collin model, D-85560 Ebersberg) at a roll temperature of 160° C. (each roll) in accordance with the following:

a) hot-rolling for 6 minutes (rolled sheet turned every minute, roll nip 0.35 mm).

| | L | a* | b* | C | h |
|---|---|---|---|---|---|
| 0.5% titanium suboxide | 34.98 | −0.38 | −0.58 | 0.69 | 236.43 |
| (based on Kronos ® 1001, | | | | | |
| example 1b)) | | | | | |
| 1.0% titanium suboxide | 29.16 | −0.53 | −3.74 | 3.78 | 261.89 |
| (based on Kronos ® 1001, | | | | | |
| example 1b)) | | | | | |

### Example 2

### Application of Pigment Mixture in PVC

0.400 g of the mica coated with tin oxide and TiO₂ layered pigment composition and 0.400 g of titanium suboxides product formed in Example 1 a) is mixed with 26.6 g of a polyviynyl chloride (PVC Evipol ® SH 7020, EVC GmbH, Frankfurt a.M), and 14.6 ml of a masterbatch consisting of
92.21% by weight of diisodecyl phthalate (Vestinol ®, Hüls Chemie)
3.60% by weight of heat stabilisers based on barium zinc carboxylate (Irgastab ® BZ561, Ciba Specialty Chemicals Inc.) and
4.19% of epoxidised Soybean oil (Rheoplast ® 39, Ciba Specialty Chemicals Inc.)

After a wetting time of 30 minutes, the mixture is processed on a roll mill for 8 minutes at a roll temperature of 160° C. to a thin strong metallic aspect film.

### Example 3

### Nail Lacquer

A rutile TiO₂ coated mica pigment is mixed with the product of example 1 a) and 1 b) in a 5:1 weight ratio and incorporated into a no-colored nail lacquer base at 5%.

Draw downs of the resulting colored nail lacquer formulation are prepared on black and white Leneta draw-down cards.

### Example 4

### Application on Skin

A rutile TiO₂ coated mica pigment is blended with the product of example 1 b) in a 2:1 and a 1:2 weight ratio and applied directly on skin, mimicking high color load of powder compositions (e.g. eye shadows).

The shade impression is silver-metallic.

### Example 5

### Application in Liquid Body Cleansing Products

The rutile TiO₂ coated mica pigment and product of example 1(b) are incorporated into an uncolored shampoo formulation at 0.05%. The mixed pigment displays a silver-gray color.

### Example 6

### Preparation of a Lipstick, Non-Greasy

| Ingredient | (w/w) % |
|---|---|
| Carnauba wax | 2.5 |
| Beeswax, white | 20.0 |
| Ozekerite | 10.0 |
| Lanoline, anhydrous | 5.0 |
| Cetyl alcohol | 2.0 |
| Liquid paraffin | 3.0 |
| Isopropyl Myristate | 3.0 |
| Propylene glycol recinoleate | 4.0 |
| CI Pigment Red 4 | 8.0 |
| Product of example 1 b) and TiO₂/Fe₂O₃ coated mica | 2.0 |
| (product of example 1 b):coated mica in a 1:1 wt. ratio) | |
| Stabilizer | 0.1 |
| Castor Oil | ad 100 |

### Example 7

### Preparation of a Lipstick, Transfer Resistant

| Ingredients | (w/w) % |
|---|---|
| Cyclomethicone | 41.50 |
| Isodecane | 10.00 |
| D&C Red No. 7 Al Lake | 5.00 |
| Synthetic wax | 6.00 |
| Isostearyltrimethylpropane siloxysilicate | 5.00 |
| Cetylstearate/acetylated lanolin, 90:10 | 5.00 |
| Ceresin | 4.00 |
| Paraffin | 3.00 |
| Product of Example 1 b) and red TiO₂-coated mica | 5.00 |
| in a 1:2 wt. ratio | |
| Methylparaben | 0.30 |
| Propylparaben | 0.10 |
| Stabilizers | 0.20 |

### Example 8

### Preparation of a Pressed Powder

| | (w/w) |
|---|---|
| Ingredients | % |
| Talc Micro Ace P-2 | 41.5 |
| Zinc Stearate | 5.0 |
| Microslip 519 (PTFE) | 4.0 |
| Nylon-12 | 4.0 |
| Sericite PHN | 10.0 |
| Ultamarine Blue | 5.0 |
| Manganese Violet | 10.0 |
| Ferric Ferrocyanide | 0.5 |
| Squalane | 5.0 |
| Product of Example 1 b) and blue TiO₂ | 14.0 |
| coated mica in a 1:2 wt. ratio | |
| Optiphen Plus | 1.0 |
| Perfume | q.s. |

### Example 9

### Preparation of a Foundation Cream

| Ingredients | (w/w) % |
|---|---|
| Titanium dioxide | 12.79 |
| Oleyl alcohol | 4.57 |
| Glyceryl stearate | 3.65 |
| Propylene glycol | 3.65 |
| Stearic acid | 1.83 |
| Magnesium aluminium silicate | 0.91 |
| Triethanolamine 99% | 0.91 |
| Iron Oxide Yellow | 0.64 |
| Fe₂O₃ coated mica (bronze color) | 0.32 |
| Cl Pigment Brown 6 | 0.37 |
| Carboxymethyl cellulose | 0.10 |
| Product of Example 1 b) | 1.00 |
| Water | ad 100 |

### Example 10

### Preparation of a Nail Varnish

| Ingredients | (w/w) % |
|---|---|
| Poly(1-trimethylsilylpropylene) | 0.30 |
| Nitrocellulose | 12.00 |
| Alkyd resin | 10.00 |
| Dibutyl phthalate | 4.00 |
| Camphor | 2.00 |
| Butyl acetate | 45.00 |
| Toluene | 20.00 |
| TiO₂ coated mica | 0.70 |
| Quaternary bentonite | 1.00 |
| Product of Example 1 a) | 5.00 |

### Example 11

### Preparation of a Shampoo:

| | (w/w) |
|---|---|
| Ingredients | % |
| Water | To 100 |
| Sodium Laureth Sulfate | 15.0 |
| Cocamidopropyl Betaine | 4.0 |
| Polyquaternium-7 | 0.4 |
| Phenoxyethanol (and) Methylparaben (and) Ethylparaben (and) | 0.5 |
| Butylparaben (and) Propylparaben (and) Isobutylparaben | |
| Citric Acid | To |
| | pH 6.5 |
| Sodium Chloride | 1.0 |
| Sodium Benzotriazolyl Butylphenol Sulfonate | 0.03 |
| Product of Example 1 a) and BiOCl platelets in a 1:2 wt. ratio | 0.05 |

### Example 11

40 g TiO₂ coated mica—CIBA XYMARA™ Silver Pearl S03 (ex CIBA SC)—and 10 g of particles obtained according to Example 1 are dispersed together in 500 ml water. The suspension is heated to 75° C. A solution of 1.96 g AlCl₃ dissolved in 200 ml water is added at 4 ml/min with a dosing pump. The suspension is maintained at pH 3 by compensation of the pH with a solution of NaOH (at 1%). The suspension is kept for 30 min at pH 3. Then the pH of the suspension is increased to pH 7 with the 1% NaOH solution. The suspension is kept for 30 min at pH 7. A solution of 0.5 g CIBA EFKA 8512 (100% active substance) dissolved in 50 ml water is added. The suspension is then kept for 60 min at pH 7.

The suspension is filtrated; the pigment is recovered, washed with water and dried overnight at 120° C. in an oven.

### Example 12

40 g TiO₂ coated mica—CIBA XYMARA™ Silver Pearl S03 (ex CIBA SC)—and 10g of particles obtained according to Example 1 are dispersed together in 500 ml water. The suspension is heated to 75° C. 6.25 grams of a DISPEX N40 (ex CIBA SC) were added to the suspension. The pH of the suspension is 9.4. After good dispersion, the pH is decreased by addition of HCl 3.5% solution to a value of 3.0. The suspension is then kept for 30 min at this pH and temperature. The suspension is filtrated; the pigment is recovered, washed with water and dried overnight at 120° C. in an oven.

Additional advantages and modifications will readily occur to those skilled in the art. Therefore, the invention in its broader aspects is not limited to the specific details and illustrative examples shown and described herein. Accordingly, various modifications may be made without departing from the spirit or scope of the general inventive concept as defined by the appended claims and their equivalents.

1. A pigment mixture comprising two different particulate components A and B, wherein component A is a spherical particle, comprising at least a titanium suboxide, a titanium oxynitride or a titanium nitride a species or mixture of species thereof; and
component B particle is an effect pigment selected from a pearlescent pigment, a metal effect pigment, an interference pigment and a luster pigment.

2. The pigment mixture according to claim 1, wherein Component A further contains unreduced TiO₂.

3. The pigment mixture according to claim 1, wherein the titanium suboxide species of component A is defined as wholly or partially reduced TiO₂ with an oxidation state of the titanium: from <4 to 2.

4. The pigment mixture according to claim 1, wherein the particle size of Component A ranges from about 30 nm to about 30 μm.

5. The pigment mixture according to claim 1, wherein the pigment mixture has a bluish hue shift, wherein the hue difference (delta b) is expressed as a CIELAB quantity and is negative.

6. The pigment mixture according to claim 1, wherein the component B is a platelet-like particle and has a (multilayer) structure comprising at least:

| | | | |
|---|---|---|---|
| TRASUB | TiO₂ | | |
| TRASUB | TiO₂ | Fe₂O₃ | |
| TRASUB | TiO₂ | Fe₃O₄ | |
| TRASUB | titanium suboxide(s) | | |
| TRASUB | TiO₂ | TiN | |
| TRASUB | TiO₂ | SiO₂ | |
| TRASUB | TiO₂ | titanium | |
| | | suboxide(s) | |
| TRASUB | TiO₂ | TiON | TiN |
| TRASUB | TiO₂ | SiO₂ | TiO₂ |
| TRASUB | TiO₂ | SiO₂ | silicon suboxide(s) |
| TRASUB | TiO₂ | SiO₂ | Fe₂O₃ |
| TRASUB | TiO₂ | SiO₂ | TiO₂/Fe₂O₃ |
| TRASUB | TiO₂ | SiO₂ | (Sn,Sb)O₂ |
| TRASUB | SnO₂ | TiO₂ | |
| TRASUB | SnO₂ | TiO₂ | Fe₂O₃ |
| TRASUB | (Sn,Sb)O₂ | SiO₂ | TiO₂ |
| TRASUB | Fe₂O₃ | SiO₂ | (Sn,Sb)O₂ |
| TRASUB | TiO₂/Fe₂O₃ | SiO₂ | TiO₂/Fe₂O₃ |
| TRASUB | Cr₂O₃ | SiO₂ | TiO₂ |
| TRASUB | Fe₂O₃ | | |
| TRASUB | Fe₂O₃ | SiO₂ | TiO₂ |
| TRASUB | titanium suboxide(s) | SiO₂ | titanium suboxide(s) |
| TRASUB | TiO₂ | SiO₂ | TiO₂ + SiO₂ + TiO₂ |
| TRASUB | TiO₂ + SiO₂ + TiO₂ | SiO₂ | TiO₂ + SiO₂ + TiO₂ |
| TRASUB | TiO₂ | Al₂O₃ | TiO₂ |
| TRASUB | Fe₂TiO₅ | SiO₂ | TiO₂ |
| TRASUB | TiO₂ | SiO₂ | Fe₂TiO₅/TiO₂ |
| TRASUB | TiO₂ | SiO₂ | MoS₂ |
| TRASUB | TiO₂ | SiO₂ | Cr₂O₃ |
| TRASUB | TiO₂ | SiO₂ | TiO₂ + SiO₂ + TiO₂+ |
| | | | Prussian Blue |
| TRASUB | TiO₂ | STL | |

wherein TRASUB is a semitransparent, or transparent substrate having a low index of refraction selected from the group consisting of natural, or synthetic mica, another layered silicate, glass, Al₂O₃, SiO_{z}, SiO₂, SiO₂/SiOₓ/SiO₂ (0.03≦x≦0.95), SiO_{1.40-2.0}/SiO_{0.70-0.99}/SiO_{1.40-2.0}, or Si/SiO_{z} with 0.70≦z≦2.0, and
STL is a semi-transparent layer selected from the group consisting of a semi-transparent metal layer of Cu, Ag, Cr, or Sn, or a semi-transparent silicon suboxide(s), titanium suboxide(s) or carbon layer.

7. The pigment mixture according to claim 1, wherein Component A is spherical and Component B is platelet-like and the diameter ratio of Component A:Component B is at least 1:2.

8. The pigment mixture according to claim 1, wherein Component A further contains unreduced TiO₂.

9. A process for producing Component A as defined in claim 1, comprising at least a titanium suboxide, a titanium oxynitride or a titanium nitride a species or mixture of species thereof, wherein the process comprises treating TiO₂ particles using a plasma torch.

10. A coating, varnish, a plastic, a paint, a printing ink, a masterbatch, a ceramic or glass, cosmetic or personal care product, comprising the pigment mixture according to claim 1.

11. A method of forming a pigment mixture comprising the steps of:
reducing TiO₂ particles to form a Component A particle, and
applying or coating said Component A particle onto a platelet-like Component B, which is different than Component A, Component A and B are as defined in claim 1.

12. The method according to claim 11, wherein Component A is spherical and Component B is platelet-like and the diameter ratio of Component A:Component B is at least 1:2.
